(19) United States
(12) Patent Application Publication
(10) Pub. No.: US 2013/0188779 A1
(43) Pub. Date: Jul. 25, 2013

(54) DEVICES, APPARATUS AND METHODS FOR ANALYZING, AFFECTING AND/OR TREATING ONE OR MORE ANATOMICAL STRUCTURES

(71) Applicants: K. S. Clifford Chao, New York, NY (US); John C. Cheeseborough, New York, NY (US); Hansen Chen, River Vale, NJ (US); Jenghwa Chang, New York, NY (US)

(72) Inventors: K. S. Clifford Chao, New York, NY (US); John C. Cheeseborough, New York, NY (US); Hansen Chen, River Vale, NJ (US); Jenghwa Chang, New York, NY (US)

(73) Assignees: Weill Cornell Medical College, New York, NY (US); The Trustees of Columbia University in the City of New York, New York, NY (US)

(21) Appl. No.: 13/575,814

(22) PCT Filed: Jan. 28, 2011
(86) PCT No.: PCT/US11/23037

§ 371 Date: Mar. 14, 2013

Related U.S. Application Data
(60) Provisional application No. 61/299,781, filed on Jan. 29, 2010.
(60) Provisional application No. 61/314,004, filed on Mar. 15, 2010.
(60) Provisional application No. 61/380,908, filed on Sep. 8, 2010.

(51) Int. Cl. A61N 5/01; A61B 8/08

(52) U.S. Cl. CPC A61N 5/01; A61B 8/085
USPC 378/150; 600/407; 600/438

(57) ABSTRACT

Exemplary embodiments of an apparatus and a system for generating at least one radiation are provided. For example, an exemplary system can include at least one radiation source arrangement configured to generate the at least one radiation, is translatable on at least one track, and is pivotable about at least two axes; a tracking assembly configured to track an internal movement of a target tissue within a living structure and provide tracking information to facilitate directing the radiation(s) generated by the radiation source arrangement(s) at the target tissue; and a collimator assembly configured to move in a curved path so as to shape and direct the at least one radiation generated by the radiation source arrangement(s).

### CROSS-REFERENCE TO PRIOR APPLICATIONS

This application claims priority to U.S. Provisional Application Ser. No. 61/299,781, filed on Jan. 29, 2010; U.S. Provisional Application Ser. No. 61/314,004, filed on Mar. 15, 2010; and U.S. Provisional Application Ser. No. 61/380,908, filed on Sep. 8, 2010. The entire disclosures of the above-referenced applications are incorporated by reference herein in their entireties.

### FIELD OF THE DISCLOSURE

The present disclosure relates to affecting anatomical structures, and more particularly, to exemplary embodiments of an apparatus and method for effectively applying radiation to an anatomical structure.

### BACKGROUND INFORMATION

Radiation therapy is an important component of cancer treatments; however, targeting radiation delivery to tumors while minimizing damage to other tissues is often a challenge. Accordingly, existing radiation therapy devices employ various configurations and designs in an attempt to precisely aim the radiation at the targeted tissue. For example, U.S. Pat. No. 7,526,066 describes a radiation therapy system for treating breasts and extremities that includes a rotating table on which the patient lies. U.S. Pat. No. 7,519,149 describes a radiation therapy machine including a linear accelerator connected to waveguide and shutter assembly configured to rotate along a rotating plane. Further, this entire assembly can be rotated in an additional direction so that the entire rotating plane can be rotated from a vertical orientation.

Additional radiation treatment apparatuses are described in U.S. Pat. Nos. 7,188,999 and 7,085,347. The apparatuses described therein are large machines which include a rotating guide that ensures that the radiation crosses an isocenter of the defined orbit, and a swinging head assembly that includes various arms and linkages. Another approach, as described in U.S. Pat. No. 7,469,035, has been described, e.g., to position the leafs of a multi-leaf collimator (MLC) during a radiation treatment.

Such prior systems have various deficiencies, which can be at least partially addressed with the exemplary devices, apparatus and methods described herein.

### SUMMARY OF EXEMPLARY EMBODIMENTS

According to one exemplary embodiment of the present disclosure, an apparatus can be provided that can be structured to tilt a support structure/track thereof at particular angles with respect to a plane of patient's body to facilitate an implementation of an irradiation via electro-magnetic radiation (e.g., X-ray therapy) to a particular target area of the patient, e.g., without impacting other healthy tissues. For example, it can be preferable to facilitate a tilt of the structure/track at an angle of over approximately 40 degrees, and more preferably between 50 and 70 degrees. Such exemplary configuration of the apparatus can be beneficial over other devices which facilitate a very slight tilt adjustment since a shifting of the weight of the conventional structures/tracts can exert an extra pressure on the support of the structure to possibly affect the stability thereof.

According to another exemplary embodiment of the present disclosure, an apparatus can also be provided which can include a mechanical apparatus/arrangement connected to the head of an linear accelerator so as to mechanically move the head in the plane which is normal to the beam direction of the resulting electro-magnetic radiation (e.g., X-ray beam) exiting the accelerator. The accelerator can be anchored at least one point, and possible up to three points. Using such exemplary apparatus, it is possible to control the direction of the irradiation, such as the X-ray beam, to a target area in any point on a plane thereof that is normal to the direction of the irradiation beam and/or pulse for a distance of up to, e.g., about 50 cm and more. The mechanical apparatus can be controlled using a computer and software executed thereby to accurately point the accelerator in the appropriate direction and toward a desired target area.

In yet another exemplary embodiment of the present disclosure, the exemplary apparatus can include an accelerator which operates at, e.g., under about 6 MV and likely at or near 2 MV, or 1 MV, which can provide the electro-magnetic radiation at low dosages and at higher dosages, e.g., controlled by a computer and/or an operator. The exemplary apparatus can include image collectors at an opposite side of the accelerator (e.g., across from the target area of the sample) which can be configured to collect the radiation from at least a portion of the target area corresponding to image of the target area of the sample. Thus, the accelerator can be placed unto a low dosage mode, and can provide such radiation to and/or through the target area. Then, the exiting radiation can be collected, and the resulting signals based on such collected radiation can be provided data to the computer for a determination of the shape and dimensions of the target area and surrounding healthy organs (and possibly for imaging one or more portions thereof). Using such information, the exemplary apparatus/device, in a high dosage mode, can irradiate the target area, e.g., selectively, and preferably only in the region which is identified as being the target region to be irradiated by the computer. Further, the imaging arrangement and/or device(s) can also collect signals from the target area during the irradiation to estimate the dose and the location of the radiation received within the patients in real time to facilitate an accurate radiation delivery.

According to yet another exemplary embodiment of the present disclosure, an apparatus can be provided in conjunction with a support, such as, e.g., a chair and/or table, which can be arranged at a tilt (e.g., between 0 and 10 degrees) with respect to the floor of the room, and which can include one or more openings thereon so that breast(s) can be extended there through. Irradiation beam (e.g., X-ray beam) source arrangement(s) can be provided at the edges of the space where the breast(s) extend, along with a shielding thereof provided on the opposite edge thereof to prevent unwanted radiation from being exposed onto the patient. The radiation (e.g., X-ray beams) can be directed to provide the energy along the plane of the table on which the body of the patient is positioned. Preferably, due to the design of the table and/or chair, the patient's knees can support the torso of the patient so as to maintain the patient's torso in the tilted position with respect to the floor. In this exemplary manner, the radiation can be directed preferably only to the affected and target areas, and the exposure of other healthy tissues is minimized.

According to another exemplary embodiment of the present disclosure, an apparatus for generating at least one radiation can be provided. The exemplary apparatus can include at least one radiation source arrangement that can be pivotable about at least two axes and can be translatable on at least one track. The axes can be substantially orthogonal to one another, and one of the two axes can be parallel to a tangent to a path of travel defined by the track. Further, the track can include a first rail and a second rail, and the radiation source arrangement can include a source arrangement disposed on the first rail and an aiming arrangement disposed on the second rail. The aiming arrangement can include first and second motors configured to pivot the radiation source arrangement about each of the two axes. Additionally, the track can have a circular shape or a non-circular shape, and the track having the non-circular shape can have at least two portions that can be substantially parallel to one another.

Further, the track can be pivotable between a first position and a second position. An angle provided between a plane defined by the track(s) in the first position and a further plane defined by the track(s) in the second position can be between about 0 degrees and about 90 degrees.

The radiation can include a radiotherapy radiation, and further can include an associated energy of about 1 MV to about 6 MV.

According to yet another exemplary embodiment of the present disclosure, another exemplary apparatus for generating at least one radiation can be provided. The exemplary apparatus can include at least one radiation source arrangement disposed on at least one track that can be pivotable between a first position and a second position. An angle provided between a plane defined by the track(s) in the first position and a further plane defined by the track(s) in the second position can be between about 0 degrees and about 90 degrees. Further, the radiation source arrangement can be pivotable about at least two axes, which can be substantially orthogonal to one another, and one of the two axes can be parallel to a tangent to a path of travel defined by the track(s). Additionally, the track can have a circular shape or a non-circular shape, and the track having the non-circular shape can have at least two portions that can be substantially parallel to one another.

The track of the exemplary apparatus can include a first rail and a second rail, and the radiation source arrangement can include a source arrangement disposed on the first rail and an aiming arrangement disposed on the second rail. The aiming arrangement can include first and second motors configured to pivot the radiation source arrangement about each of the at least two axes. Further, the radiation can include a radiotherapy radiation, and can also include an associated energy of about 1 MV to about 6 MV.

According to yet another exemplary embodiment of the present disclosure, yet another exemplary apparatus for generating at least one radiation can be provided. The exemplary apparatus can include at least one radiation source arrangement having a collimator assembly which can be configured to move in a curved path so as to shape and direct radiation(s) generated by the radiation source arrangement(s). The collimator assembly can include a plurality of jaws movable about a first axis, and a further plurality of jaws rotatable about a second axis, where the first and second axes can be substantially orthogonal to one another. Further, the plurality of jaws can be movable in a curved manner in opposite directions and simultaneously in a same direction. Additionally, the radiation can provided through an opening created by the plurality and further plurality of jaws. The radiation can include a radiotherapy radiation, and can also include an associated energy of about 1 MV to about 6 MV.

According to yet another exemplary embodiment of the present disclosure, an apparatus for tracking a target tissue can be provided. The exemplary apparatus can include a tracking assembly configured to track an internal movement of the target tissue within a living structure. The tracking assembly can include an ultrasonic imaging device that can be configured to track the movement of the target tissue by continuously acquiring a position of a tissue associated with the target tissue. For example, the tissue associated with the target tissue includes a diaphragm.

The exemplary apparatus can also include at least one radiation source arrangement which is configured to generate at least one radiation directed at the target tissue and directing the radiation(s) at the target tissue at least partially based on tracking information provided by the tracking assembly based on the movement. The radiation source arrangement can be translatable on at least one track and can be pivotable about at least two axes. One of the two axes can be parallel to a tangent to a path of travel defined by the track(s), and the track can be pivotable between a first position and a second position. An angle provided between a plane defined by the track(s) in the first position and a further plane defined by the track(s) in the second position can be between about 0 degrees and about 90 degrees. Further, the track can include a first rail and a second rail, and the radiation source arrangement can include a source arrangement disposed on the first rail and an aiming arrangement disposed on the second rail. The aiming arrangement can include first and second motors configured to pivot the radiation source arrangement(s) about each of the at least two axes.

Further, the tracking assembly can provide the tracking information dynamically in real-time.

The exemplary apparatus can also include a collimator assembly configured to move in a curved path so as to shape and direct the radiation generated by the radiation source arrangement. The collimator assembly can include a plurality of jaws movable about a first axis and a further plurality of jaws rotatable about a second axis.

According to yet another exemplary embodiment of the present disclosure, a system for generating at least one radiation can be provided. The exemplary system can include at least one radiation source arrangement configured to generate the radiation(s), which can be translatable on at least one track and pivotable about at least two axes; a tracking assembly configured to track an internal movement of a target tissue within a living structure and provide tracking information to facilitate directing the radiation(s) generated by the radiation source arrangement(s) at the target tissue; and a collimator assembly configured to move in a curved path so as to shape and direct the radiation(s) generated by the radiation source arrangement(s). The track can be pivotable between a first position and a second position, and an angle provided between a plane defined by the track(s) in the first position and a further plane defined by the track(s) in the second position can be between about 0 degrees and about 90 degrees.

Further, the two axes can be substantially orthogonal to one another, and one of the two axes can be parallel to a tangent to a path of travel defined by the track(s). Additionally, can include a first rail and a second rail, and the radiation source arrangement can include a source arrangement disposed on the first rail and an aiming arrangement disposed on the second rail. The aiming arrangement can include first and second motors configured to pivot the radiation source arrangement(s) about each of the at least two axes.

The track can have a circular shape or a non-circular shape, and the tracking having the non-circular shape can include at least two portions that can be substantially parallel to one another.

Further, the collimator assembly can include a plurality of jaws rotatable about a first axis and a further plurality of jaws rotatable about a second axis. The first and second axes can be substantial orthogonal to each other.

The radiation can include a radiotherapy radiation and can include an associated energy of about 1 MV to about 6 MV.

Additionally, the tracking assembly can include an ultrasonic imaging device configured to track the movement of the target tissue by continuously acquiring a position of a tissue associated with the target tissue, and the radiation source arrangement can generate the radiation directed at the target tissue and can direct the radiation(s) at the target tissue based on tracking information provided by the tracking assembly. Further, the tracking assembly can provide tracking information dynamically in real-time, and in an exemplary embodiment, for example, the tissue associated with the target tissue includes a diaphragm.

The exemplary system can further include an optical surface tracking arrangement, which can include a plurality of optical sensors disposed radially around an opening configured to receive at least a portion of a patient.

These and other objects, features and advantages of the exemplary embodiments of the present disclosure will become apparent upon reading the following description thereof, when taken in conjunction with the appended claims.

### BRIEF DESCRIPTION OF THE DRAWINGS

Exemplary objects, features and advantages provided by the present disclosure will become apparent from the following detailed description taken in conjunction with the accompanying figures showing illustrative embodiments, in which:

FIGS. 1A and 1B are respective top and lateral views of an apparatus for providing electro-magnetic radiation (e.g., radiotherapy) to one or more anatomical structures (e.g., a breast, etc.) according an exemplary embodiment of the present disclosure;

FIG. 2 is a perspective view of an exemplary aiming and control mechanism/arrangement of the exemplary apparatus shown in FIGS. 1A and 1B, which can include a plurality (e.g., tangent and vertical) linear stage motors at or on an outer support;

FIGS. 3A and 3B are respective side and top views of an exemplary implementation of an accelerator arrangement of the exemplary apparatus of FIGS. 1A and 1B to implement exemplary irradiation of the sample in different directions and angles according to the exemplary embodiment of the present disclosure;

FIGS. 4A and 4B are exemplary diagrams to determine a target location and direction for irradiation of the sample using the exemplary apparatus shown in FIGS. 1A and 1B;

FIG. 5A is a side view of an exemplary platform which can be used with the exemplary embodiments of the apparatus according to the present disclosure that can be lifted to create an inclination up to, e.g., 60-degree relative to the floor;

FIG. 5B is a side view of an implementation of the exemplary embodiment of the platform shown in FIG. 5A which can be used to irradiate a sample and/or anatomical structure shown therein;

FIG. 6 is a side cut-away view of an exemplary table and supporting structure according to an exemplary embodiment of the present disclosure which can be used to irradiate the sample, structure and/or at least one portion thereof;

FIG. 7 is a perspective view of an exemplary tracking apparatus and an inset tracking trace according to an exemplary embodiment of the present disclosure;

FIG. 8 is a side view of a collimator system having a primary assembly according to another exemplary embodiment of the present disclosure;

FIG. 9 is a side view of another collimator system having a primary assembly and a secondary assembly according to a further exemplary embodiment of the present disclosure;

FIG. 10 is a perspective view of the exemplary collimator system according to an exemplary embodiment of the present disclosure during an installation of the primary assembly on a radiation source;

FIG. 11 is a perspective view of the exemplary collimator system according to the exemplary embodiment of the present disclosure during an installation of an inner jaw assembly of the secondary assembly;

FIG. 12 a perspective view of the exemplary collimator system shown in FIG. 11 providing an exemplary radiation beam with a particular cross-section from the inner secondary assembly of an exemplary collimator system according to an exemplary embodiment of the present disclosure;

FIG. 13 is a front view of an exemplary embodiment of an outer jaw assembly of the secondary assembly of the collimator system according to an exemplary embodiment of the present disclosure shown in FIG. 12;

FIG. 14 is a perspective view of a radiation beam cross-section emitted from the exemplary outer secondary assembly of the exemplary collimator system shown in FIG. 12 according to an exemplary embodiment of the present disclosure during an operation thereof;

FIG. 15 is a perspective view of the exemplary collimator system according to an exemplary embodiment of the present disclosure shown in FIG. 12 providing a wide beam output, in operation;

FIG. 16 is a perspective view of the exemplary collimator system according to an exemplary embodiment of the present disclosure shown in FIG. 12 providing a narrow beam output in operation;

FIG. 17 is a side perspective view of an installed exemplary collimator system according to an exemplary embodiment of the present disclosure;

FIG. 18 is a perspective view of an exemplary radiotherapy system according to an exemplary embodiment of the present disclosure;

FIG. 19 is a perspective view of an exemplary radiotherapy system according to still another exemplary embodiment of the present disclosure;

FIGS. 20A and 20B are illustrations of the exemplary radiotherapy system in use according to further exemplary embodiments of the present disclosure;

FIG. 21 is a visual radiation illustration/simulation of an exemplary treatment using radiation beams provided by the exemplary radiotherapy system according to various exemplary embodiments of the present disclosure;

FIG. 22 is an illustration of an exemplary treatment using the exemplary radiotherapy system according to various exemplary embodiments of the present disclosure;

FIG. 23 is another illustration of a further exemplary treatment using the exemplary radiotherapy system according to various exemplary embodiments of the present disclosure;

FIG. 24 is still another illustration of yet another exemplary treatment using the exemplary radiotherapy system according to various exemplary embodiments of the present disclosure; and

FIG. 25 is an exemplary block diagram of an apparatus according to further exemplary embodiments of the present disclosure.

Throughout the figures, the same reference numerals and characters, unless otherwise stated, are used to denote like features, elements, components or portions of the illustrated embodiments. Moreover, while the present disclosure will now be described in detail with reference to the accompanying figures, it is done so in connection with the illustrative embodiments. It is intended that changes and modifications can be made to the described embodiments without departing from the true scope and spirit of the present disclosure.

DETAILED DESCRIPTION OF EXEMPLARY EMBODIMENTS

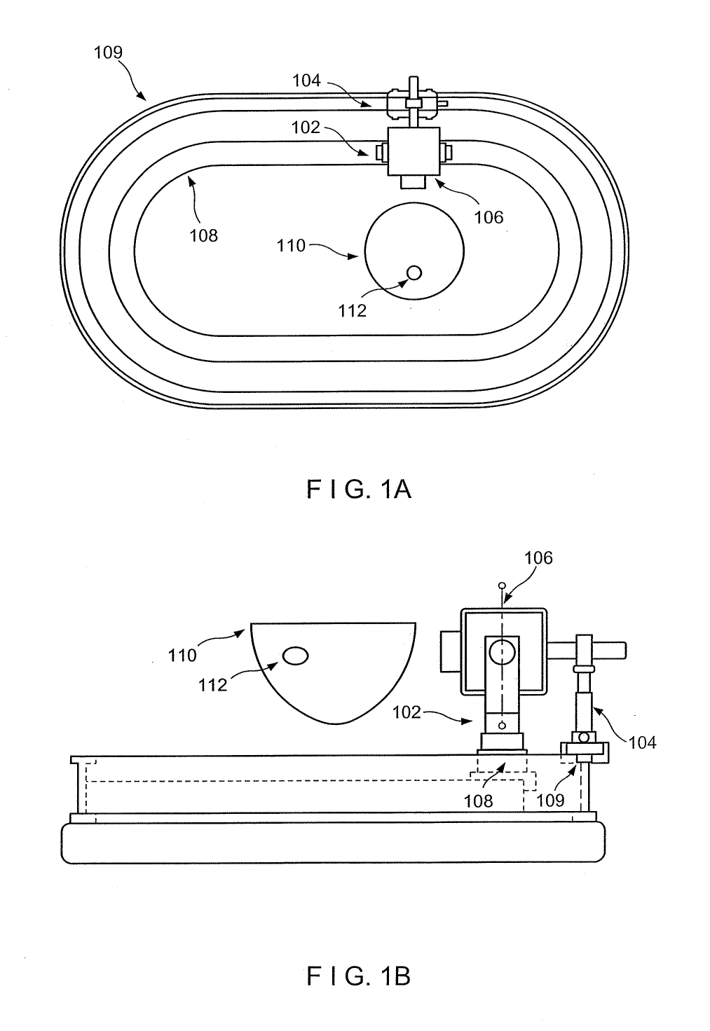

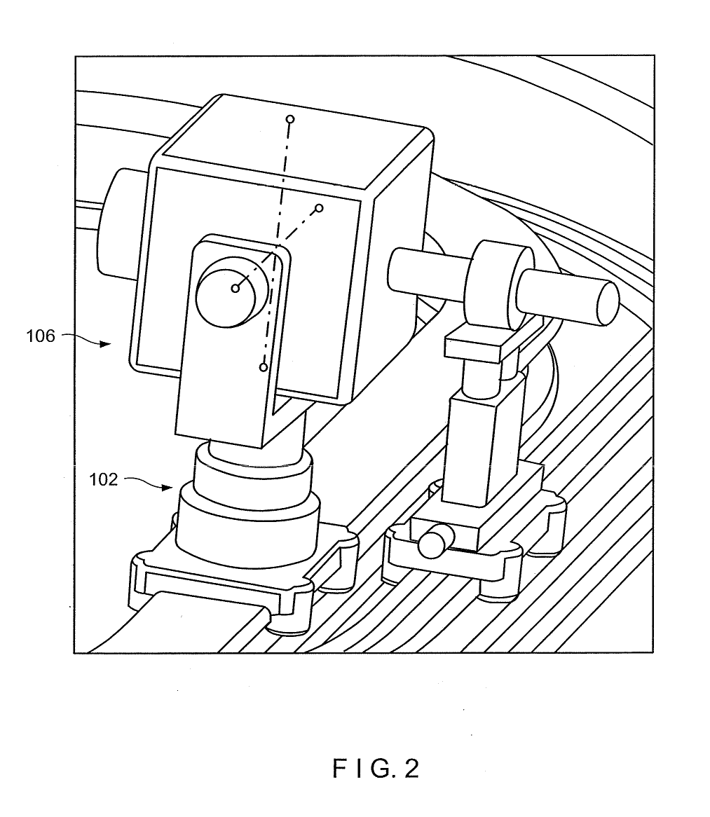

FIGS. 1A and 1B illustrate respective top and lateral views of an exemplary embodiment of an apparatus for providing electro-magnetic radiation (e.g., radiotherapy) to an anatomical structure (e.g., a breast) and/or one or more portions thereof according to the present disclosure. This exemplary apparatus can include certain structures and/or devices 102, 104 (e.g., “cars”) being translated on two rails 108, 109. The rails 108, 109 can be provided in a race track shape, but can also be of a circular shape, as well as other shapes. For example, an inner device/car 102 can be provided on an inner rail 108 for carrying a linac gun 106 and/ or other radiation generating source, and an outer device/car 104 can be provided on an outer rail 109 for the aiming device. Inner device/car 102 can also include a collimator arrangement. An object 110 shown in FIGS. 1A and 1B is the sample (e.g., a breast) and a further object 112 inside the sample 110 can be a treatment target. Both devices/cars 102, 104 can travel on a horizontal plane parallel to the floor. Because the rails 108, 109 can be non-circular, according to one exemplary embodiment, there is likely no rotation isocenter and the linac gun 106 does not point at fixed point when it travels on the inner rail 108. On the other hand, the linac gun 106 can point in the normal direction (e.g., perpendicular to the tangent) of the travel trajectory. As shown in FIGS. 1A and 1B, the tangent, normal and vertical (e.g., the outer product of the tangent and normal) vectors can constitute three axes of the local Cartesian coordinate system with the linac gun's 106 center being the origin, and the linac gun 106 can point in various directions in the three-dimensional space.

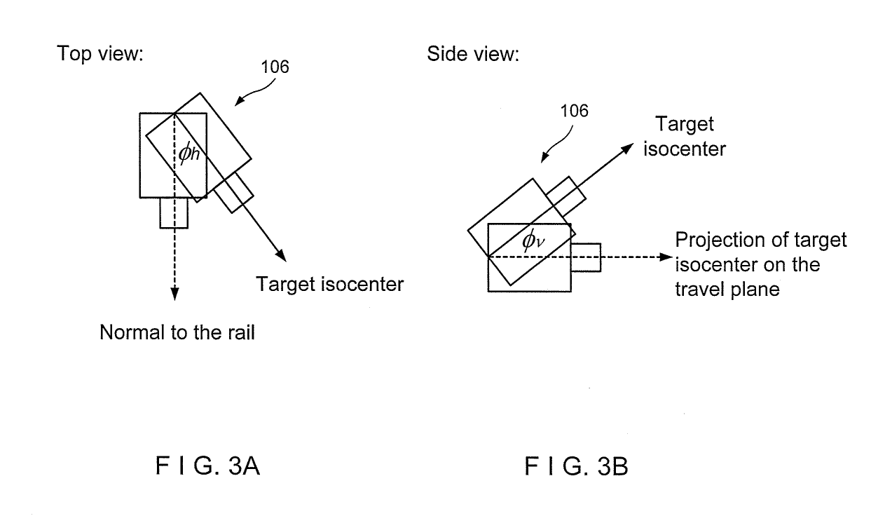

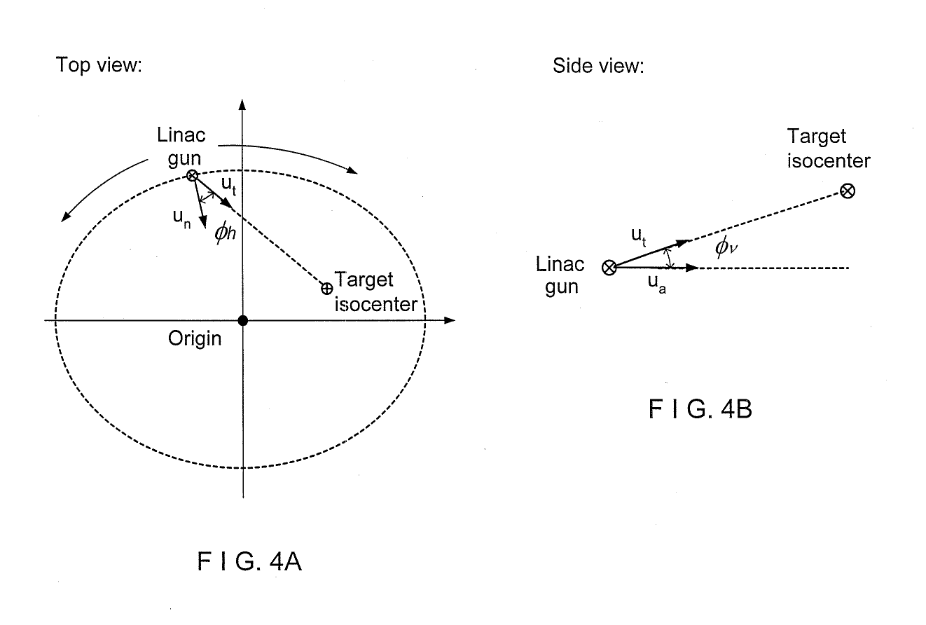

FIG. 2 illustrates an exemplary embodiment of an aiming and control mechanism of the exemplary apparatus shown in FIGS. 1A and 1B, which can include a plurality (e.g., tangent and vertical) linear stage motors on the outer car. For example, the motion of a tangent motor cause an assembly of the linac gun 106 (e.g., the inner device/car 102) to rotate around the vertical axis extending away from the inner rail 108, and the vertical motion can lead to the rotation of the assembly about the tangent axis. Thus, the linac gun 106 can have, e.g., at least three motions: e.g., traveling on the rail to a predetermined lateral location, a horizontal scan around the vertical axis; and a vertical tilt around the tangent axis. The position of the center of the linac gun 106 at any time can be determined by the sensors on the inner rail 108 and/or the outer rail 109. As shown in FIGS. 3A and 3B, according to certain exemplary embodiments of the present disclosure, it is possible to aim the linac gun 106 using such exemplary arrangements to determine the horizontal scan angle φₕ and the vertical tilt angle φᵥ for a given rail position so that the linac gun 106 preferably likely points and/or aims at the isocenter of the treatment target 112.

Mathematically, this exemplary procedure can be facilitated, as shown in the exemplary diagrams of FIG. 4, where uₙ is the unit normal vector, uₜ is the unit vector from the linac gun 106 to the isocenter of the treatment target 112, and uₐ is the unit vector from the linac gun 106 to the isocenter of the treatment target 112 projected on the travel plane. The “Origin” can be the origin for the global Cartesian coordinate system for the treatment room.

The three unit vectors uₙ, uₜ, and uₐ, as shown in FIGS. 3A and 3B can be calculated or determined (e.g., using a computing arrangement) from the global Cartesian coordinates of the center of the linac gun 106 and the isocenter of the treatment target 112. The two rotation angles of aiming can then be calculated using simple vector relations: φₕ=cos⁻¹(uₙ . uₐ) and φᵥ=cos⁻¹(uₜ . uₐ). The determined and/or calculated target angles can be compared to the current angle, and the differences can be provided to a control unit/arrangement to drive the linear stage to, e.g., rotate and point the linac gun 106 at the isocenter of the treatment target 112 in real time.

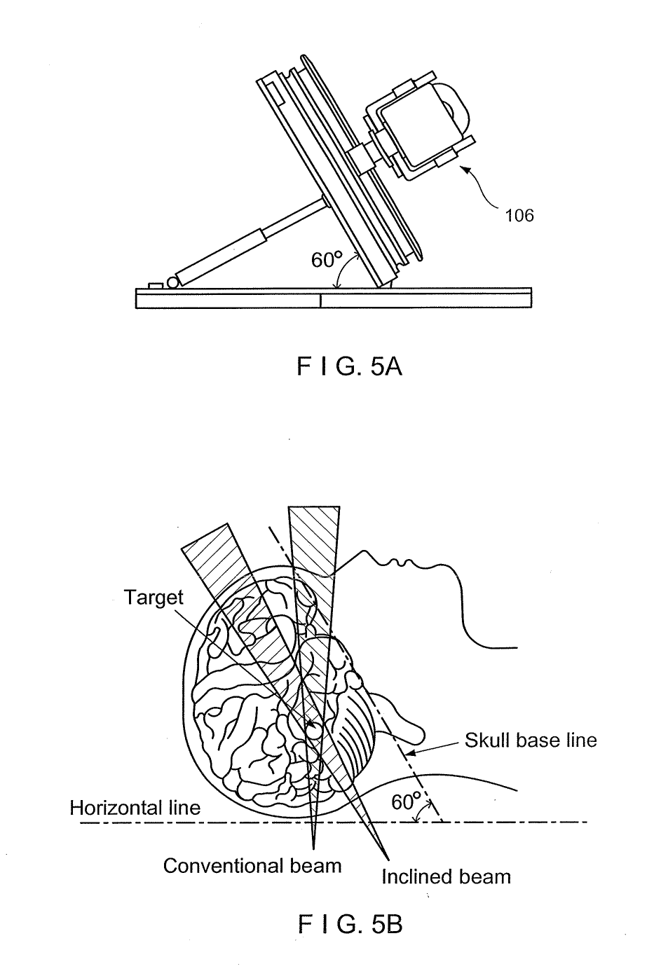

Further, according to another exemplary embodiment of the present disclosure, as shown in FIG. 5A, the main platform which houses or facilitates the assembly of the linac gun 106 can be lifted or raised to provide an exemplary inclination of, e.g., up to about 60degrees, or up to about 90-degrees, relative to the floor. This exemplary inclination can be achieved by an exemplary embodiment of a platform lift mechanism which can include, e.g., servo-mechanical linear actuators mounted to specific points on the instrument base and the main platform. An extension of the linear actuators can then lift and/or raise the main platform to an angle of inclination of, e.g., up to about 60 degrees. The exemplary angle of inclination can be obtained from, e.g., a rotary encoder installed or provided in or at the rotation axis of the main platform. In other exemplary embodiments, the platform can be lifted or raised to provide an inclination of up to about 90 degrees, relative to the floor. Accordingly, the platform lift mechanism can angle the platform anywhere from, e.g., about 0 degrees (e.g., parallel to the floor) to about 90 degrees (e.g., at a right angle to the floor), such as about 10 degrees, about 20 degrees, about 45 degrees, about 70 degrees, about 80 degrees, etc. relative to the floor.

The exemplary inclination of the main platform can serve the following exemplary purposes. For a breast treatment, e.g., a comfortable treatment position may not be a flat position, and possibly be the position with the head of a patient slightly elevated. For such exemplary case, the main platform can be lifted for about 10 degree inclination to better match the shape of the patient body so that the radiation beam does not diverge into other critical organs (e.g., heart, lung, etc.). For the brain treatment, the exemplary inclination can be, e.g., around 60 degrees. As shown in FIG. 5B, this inclination angle can be selected so as to match, at least approximately the angle between the skull base and the horizontal line. It is known that lesions in the occipital lobe and cerebellum areas are difficult to target for the brain radiotherapy using a conventional linac gun because a posterior beam (e.g., “conventional beam” shown in FIG. 5B) usually impacts the optical structures (e.g., eye, optical nerves, optical chiasm, etc.). If the radiation beam (e.g., “Inclined beam” shown in FIG. 5B) is tilted to match the skull base line, lesions in these areas can be conveniently reached by the radiation beam without involving the optical structures.

According to another exemplary embodiment of the present disclosure, the imaging device/arrangement can be provided with the radiation therapy arrangement which can include a flat panel imager that can serve dual purposes. First, e.g., the imaging radiation can travel around the path opposing to the radiation sources to produce projection images for, e.g., three-dimensional (3D) CBCT reconstruction or generate two-dimensional (2D) orthogonal pair of projection images of a portion of living organs including the treatment target. These exemplary images can be used for 3D or 2D image-guided configurations, devices and/or arrangements. The imaging device/arrangement can also be calibrated to measure the radiation dose. This capability can facilitate an estimation of the dose and location of the radiation deposited inside the patients in real time to assure accurate radiation delivery. The same radiation source can be used to provide both the imaging radiation and the therapy radiation to the sample. For example, to image the sample and locate the target therein, the radiation source can be placed into a low dosage mode, and can provide such radiation to and through the target area. Then, the exiting radiation can be collected, and the resulting signals can provide data to the computer so as to determine the shape and dimensions of the target area and surrounding healthy organs (and possibly for imaging such target area and/or one or more portions thereof). Using such exemplary information, the exemplary device, in a high dosage mode, irradiates the target area, e.g., possibly only in the region which is identified as being the region to be irradiated by the computer. Thus, the imaging device/arrangement can collect signals provided from the target area during irradiation so as to estimate the dose and location of the radiation deposited inside the patients in real time to assure accurate radiation delivery.

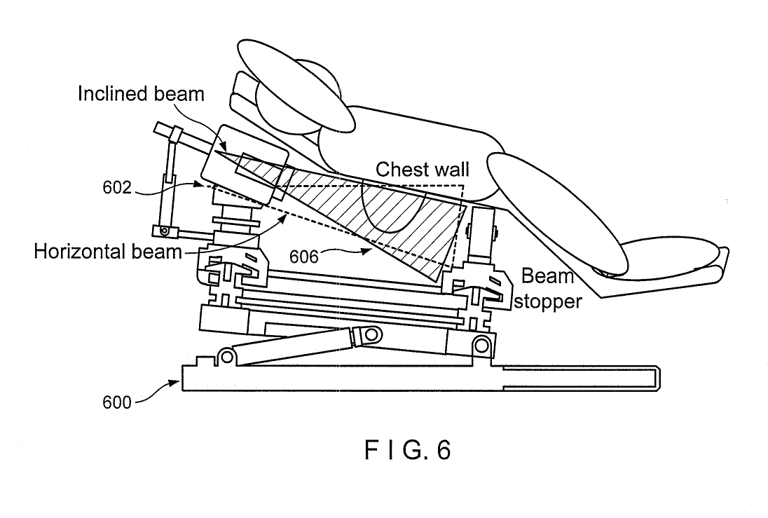

FIG. 6 shows an exemplary embodiment of a table 600 and a supporting structure according to another exemplary embodiment of the present disclosure, which can be used to irradiate the sample and/or one or more portions thereof. For example, the inclination of the exemplary table 600 shown in FIG. 6 can be utilized to implement the exemplary embodiment shown in FIGS. 5A and 5B, and described herein above. For example, as shown in FIG. 6, a horizontal beam 602, which is parallel to the floor and to the chest wall of a patient is provided if the patient's head is not raised. However, such beam would likely diverge into the patient if the exemplary embodiment of the table according to the present disclosure, as shown in this figure, is inclined to raise the patient head for a more comfortable position. Thus, in the inclined state, which is shown in FIG. 6, an inclined beam 606 parallel to the chest wall can be achieved by raising the main platform of the exemplary chair. In this exemplary manner, the beam can likely be prevented or stopped, e.g., by a beam stopper and would not diverge into the patient.

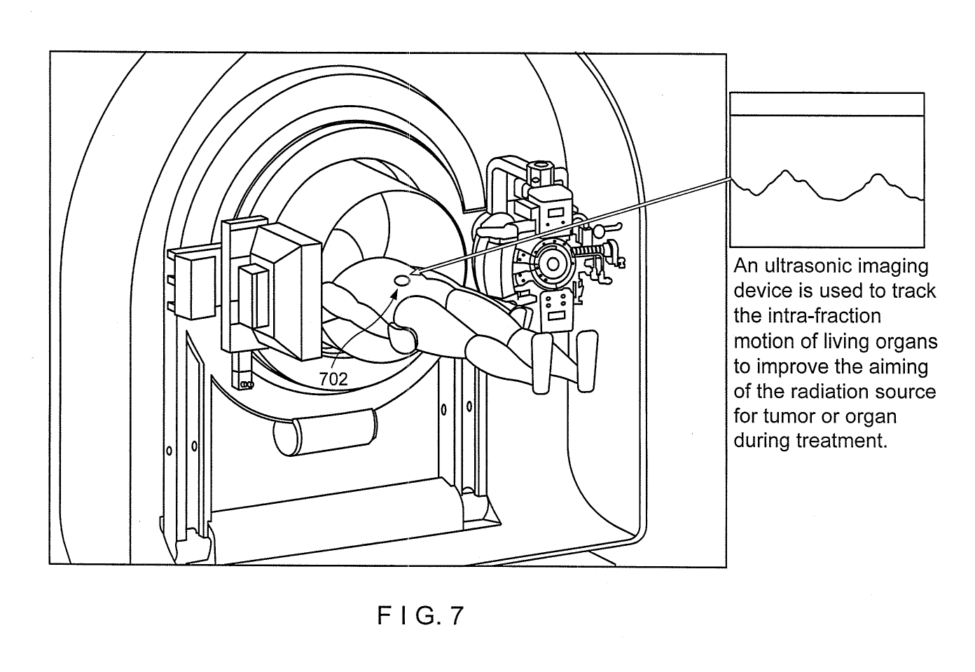

According to still another exemplary embodiment of the present disclosure, an apparatus can be provided which facilitates tracking a movement of living organs while a target is being treated in real-time. The exemplary tracking apparatus can include an imaging device that can acquire and track a position of an organ in real-time. For example, as shown in FIG. 7, the imaging device can include an ultrasonic imaging device 702, which can be attached to a patient undergoing radiotherapy. As shown in FIG. 7, the ultrasonic imaging device 702 can track, in substantially real time, a movement of the organ being targeted by the radiation (e.g., radiotherapy treatment). The exemplary ultrasonic imaging device can be used to track an intra-fraction motion of living organs to improve the aiming of a radiation source during the application and/or the treatment. For example, the movement of a person's lung can correlate to the movement of the person's diaphragm. Accordingly, if a portion of a lung is being targeted by the radiotherapy, the ultrasonic imaging device 702 can track the movement of a patient's diaphragm in order to track the movement of one or more portions of the lung being targeted. The type of motion that can be tracked can include, e.g., a movement associated with activities such as breathing, etc.

Such exemplary real-time tracking information can be provided to a control and/or aiming system which can control the targeting and/or aiming of the radiation being applied to the target area (e.g., using radiotherapy). Accordingly, the radiation beam can be adjusted and repositioned accordingly to ensure that correct tissue is being targeted during the treatment. For example, the linac gun 106 that can be used in conjunction with the exemplary apparatus described herein can be positioned and aimed based on tracking information provided by the imaging device 702. The aiming and positioning of the linac gun 106 can be effectuated by controlling the position of the linac gun 106 on the rail(s) 108, 109, and controlling the rotation of the linac gun 106 around the vertical and tangential axes.

Further, the exemplary tracking apparatus can utilize a continuous feedback arrangement so that the movement/tracking information can be provided to the control and/or targeting system in a substantially real-time manner, e.g., during the irradiation of the target area and/or the treatment thereof. Additionally or alternatively, the exemplary tracking apparatus can include a safety mechanism. For example, the exemplary tracking apparatus can be programmed with one or more pre-determined thresholds such that if a movement larger than the pre-determined threshold is detected, the application of the radiation , e.g., the radiotherapy can be ceased so that the non-targeted tissue is not inadvertently irradiated by the treatment. In one exemplary embodiment of the present disclosure, such exemplary tracking apparatus can be implemented and used in conjunction with the exemplary apparatus and system and the exemplary collimator apparatus described herein.

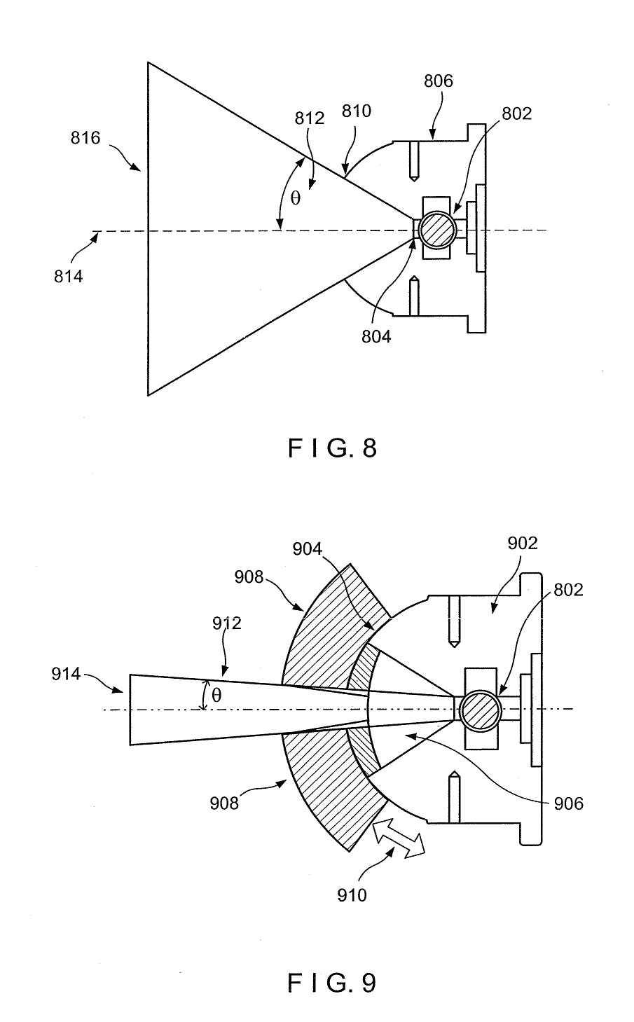

According to still another exemplary embodiment of the present disclosure, a collimator apparatus can be provided which includes, e.g., primary and secondary collimator bodies, stages and/or assemblies, as shown in FIG. 8. For example, radiation emitted from a radiation source 802 can pass through an entrance aperture 804 in a primary collimator assembly body 806, as shown in FIG. 8. The radiation can then exit the primary collimator body through an exit aperture 810. The exit aperture 810 can have the same cross-sectional shape and angular orientation as the entrance aperture 804, and according to one exemplary embodiment, can be larger in cross-sectional area than the entrance aperture 804. The size of the exit aperture 810 relative to the entrance aperture 804 can determine an angle of divergence 812 of the radiation beam exiting from the primary collimator body 806 with respect to a collimator centerline 814. Such angle 812 can establish, e.g., the maximum angle of divergence of the radiation beam that the collimator can achieve. A cross-section of a resultant radiation beam 816 can then be provided a function of its distance from the entrance aperture 804.

The secondary collimator stage/assembly can include two subassemblies. Such subassemblies can include and be referred herein to as “jaws”. For example, as shown in FIG. 9 which illustrates further exemplary embodiment of the primary and secondary collimator assemblies, the radiation emitted by the radiation source 802 can pass through a primary collimator assembly 902, exiting through an aperture 904. Only the inner “jaw” assembly is shown in FIG. 9 for clarity. Each set of jaws, or the jaw assembly, can include two members 906, 908 (or more) that can move in a curved path 910 over the aperture 904 of the primary collimator in a manner such that the members 906, 908 can at least partially obstruct the radiation beam exiting the primary collimator assembly to a greater or lesser extent. The positions of the jaws of the secondary collimator assembly can then drive or control the angle of divergence 0 of the output radiation beam 912, and thereby control the cross-sectional area of a final radiation beam 914 at a desired distance from the radiation source 802. The outer jaws of the secondary collimator stage/assembly can move in a direction that can be perpendicular to that of the inner jaws. Such exemplary configuration can produce a beam with a rectangular cross-section. It should be understood that other directions of movement of the jaws and/or its components 906, 908 are possible and are within the scope of the present disclosure.

Since the members 906, 908 of the secondary collimator assembly according to the exemplary embodiment of the present disclosure can move in a curved path over the primary collimator body, they do not require as much space when in the open position as do traditional collimators, which typically includes a secondary collimator assembly having member that move in a straight path that is more or less perpendicular to the path of the radiation beam.

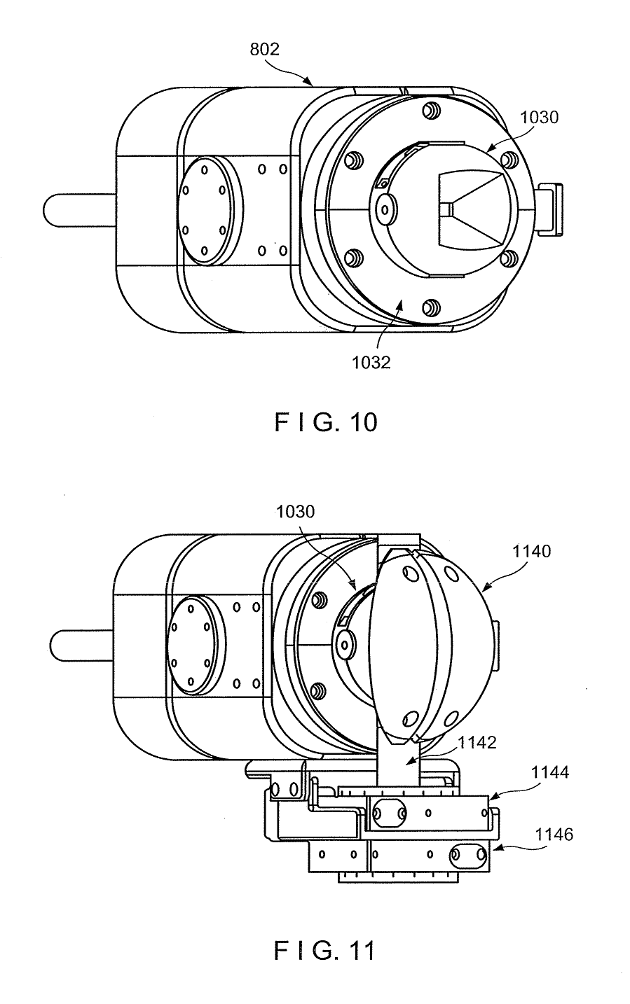

FIG. 10 shows an exemplary embodiment of a primary collimator assembly 1030 mounted to an output end of the radiation source 802 which can be, e.g., a linear accelerator. The primary collimator assembly 1030 can be installed on the radiation source via a mounting flange 1032.

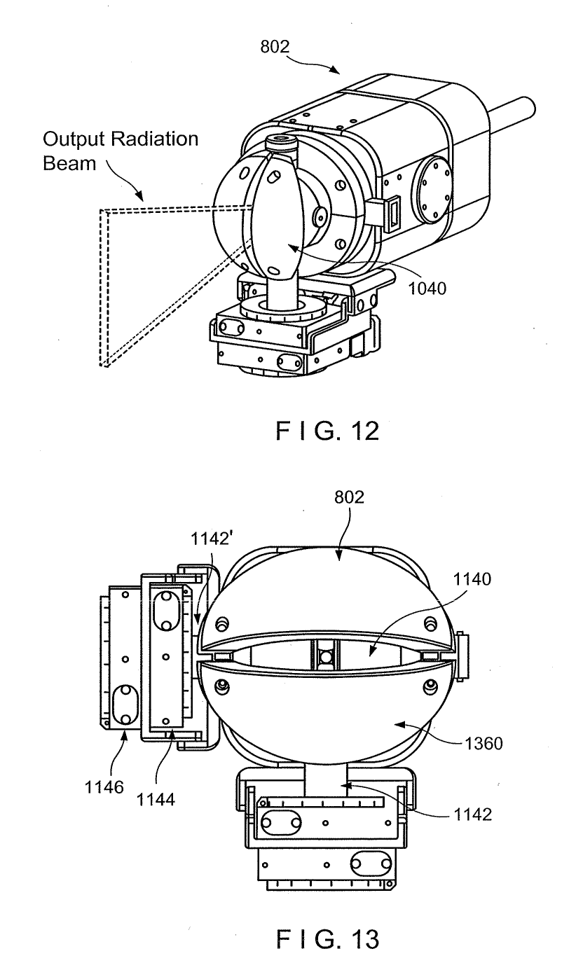

FIGS. 11 and 12 show perspective view of the primary collimator assembly 1030 mounted to the radiation source 802 provided in a closed or mostly closed configuration according an exemplary embodiment of the present disclosure. As shown in FIGS. 11 and 1, the jaws of an inner jaw assembly 1140 can be mounted to the body of the primary collimator assembly 1030. The orientation of the inner jaws can be such that they control the horizontal or “X” dimension of the beam cross-section. A rotation axes for the jaws can be implemented as a pair (or a plurality) of concentric drive shafts 1142. The drive shafts 1142 can then be connected to, e.g., computer-controlled, motorized rotary stages 44, 46, e.g., each having a center bore through which the drive shafts 42 of the inner jaw axes can pass. The rotary stages 1144, 1146 can rotate in opposite directions to open and/or close the distance between the two inner jaws. A horizontal cross-section of the radiation beam can increase as the inner jaws open, as shown in FIG. 12, and decreases as they close. It is also possible to move the jaws simultaneously in the same direction which can results in a horizontal sweeping motion of the output radiation beam.

As shown in FIG. 13, the jaws of an outer jaw assembly 1360 can also be mounted to a body of the primary collimator assembly 1030. The jaws of the outer jaw assembly 1360 can have, e.g., the same thickness as those of the inner jaw assembly 1140, and can be larger in radius so as to facilitate the outer jaw assembly 1360 to move along the curved surface of the inner jaws, underneath thereof. The orientation of the outer jaws can be such that they can control the vertical or “Y” dimension of the beam cross-section. The rotation axes for the jaws can be implemented, e.g., as a pair or a plurality of concentric drive shafts 1142. The drive shafts 1142 can then be connected to computer controlled, motorized rotary stages 1144, 1146, e.g., each having a center bore through which drive shafts of the outer jaw axes can pass. The rotary stages 1144, 1146 can rotate in opposite directions to open and/or close the distance between the outer jaws. The vertical cross-section of the radiation beam can be increased as the outer jaws open, and decreases as they close. It is also possible to move the jaws simultaneously in the same direction which can result in a vertical sweeping motion of the output radiation beam. In a further exemplary embodiment, the inner jaws can be configured such that they can be controlled in the vertical or “Y” dimension, and the outer jaws can be configured such that they can be controlled in the horizontal or “X” dimension.

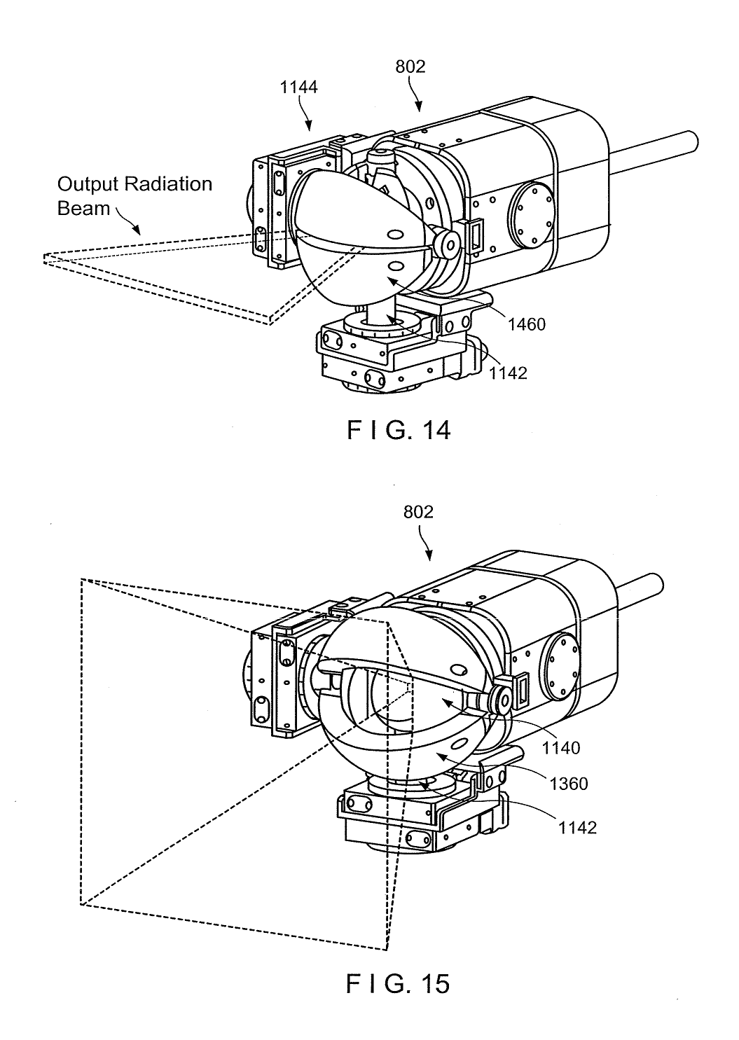

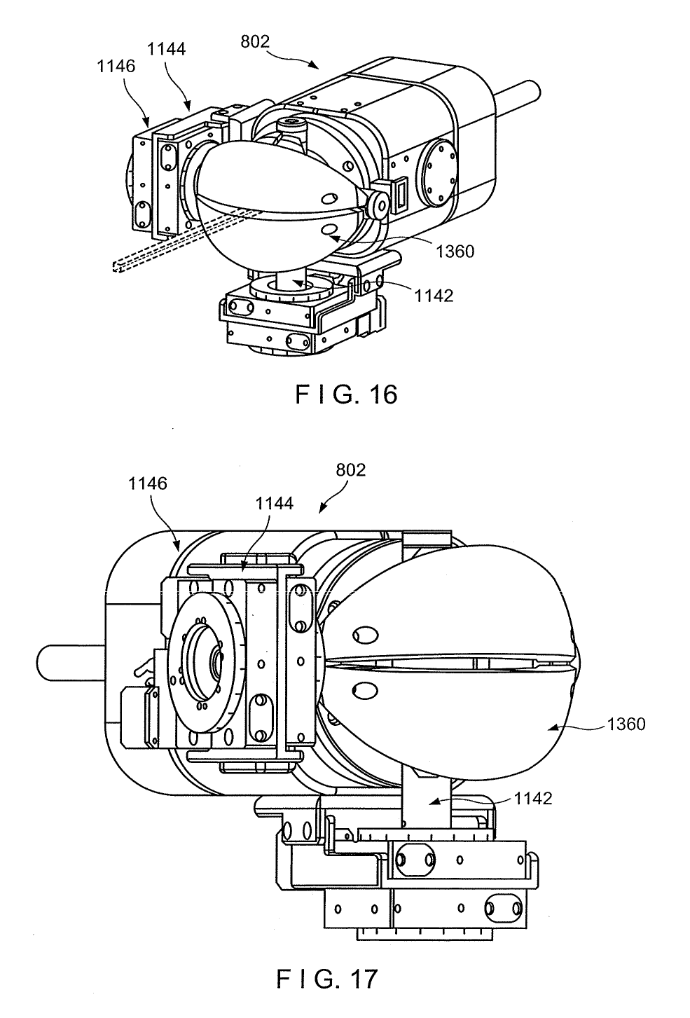

FIG. 14 shows a perspective view of the exemplary embodiment of the collimator system according to an exemplary embodiment of the present disclosure in operation, e.g., in which a narrow beam output is generated. For example, the exemplary collimator system can be configured to produce large and small output radiation beam cross- sections, and can also facilitate a propagation of the radiation at various angles while mounted to the body of the radiation source 802 using the jaws assembly 1360 and the rotary stages 1144, 1146. Further, as described above, the secondary collimator assembly can facilitate steering of the beam output so that a narrow beam can be used to sweep through a larger area to be irradiated, providing more precise and accurate irradiation. FIG. 15 shows a side perspective view of an installed collimator system according to the exemplary embodiment of the present disclosure, which can provide a wide beam due to the control of the jaws assembly 1360 and the rotary stages 1144, 1146. FIGS. 16 and 17 show a side perspective view of the collimator system according to the exemplary embodiment of the present disclosure, providing a small pin-type beam, and no beam, respective, again due to the control of the jaws assembly 1360 and the rotary stages 1144, 1146.

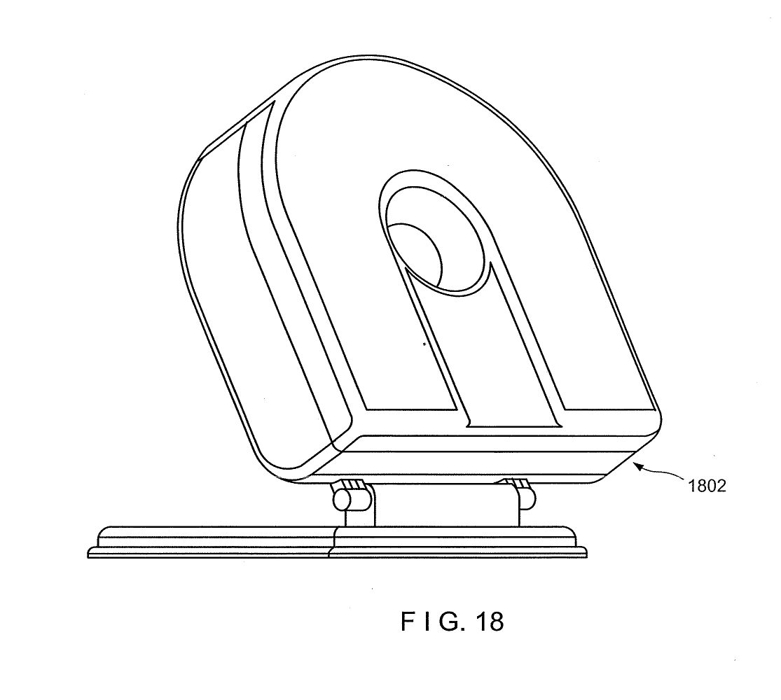

According to yet another exemplary embodiment of the present disclosure, a radiotherapy system 1802 can be provided, as shown in FIG. 18. In this exemplary embodiment, the radiotherapy system 1802 can include a compact, robotic, non-coplanar, intensity-modulated radiation therapy (IMRT) and/or image guided radiation therapy (IGRT) radiation therapy system. For example, the radiotherapy system 1802 can provide, e.g., isocentric or non-isocentric, coplanar or non-coplanar IMRT, or linear accelerator-based radiotherapy without isocentric or coplanar limitations. The exemplary radiotherapy system 1802 can include some, many or all of the exemplary features described above. For example, the exemplary radiotherapy system 1802 can include the exemplary -device/car and rail structure described above (and shown in, e.g., FIGS. 1-6) that can facilitate control, aiming, and targeting of the radiotherapy. Further, as shown in FIG. 18 and described above (and shown in, e.g., FIGS. 5 and 6), the platform and/or the housing supporting the radio therapy apparatus can be angled at an angle between about 0 degrees and about 90 degrees relative to the floor. In FIG. 18, the exemplary platform is shown at approximately an 80 degree angle.

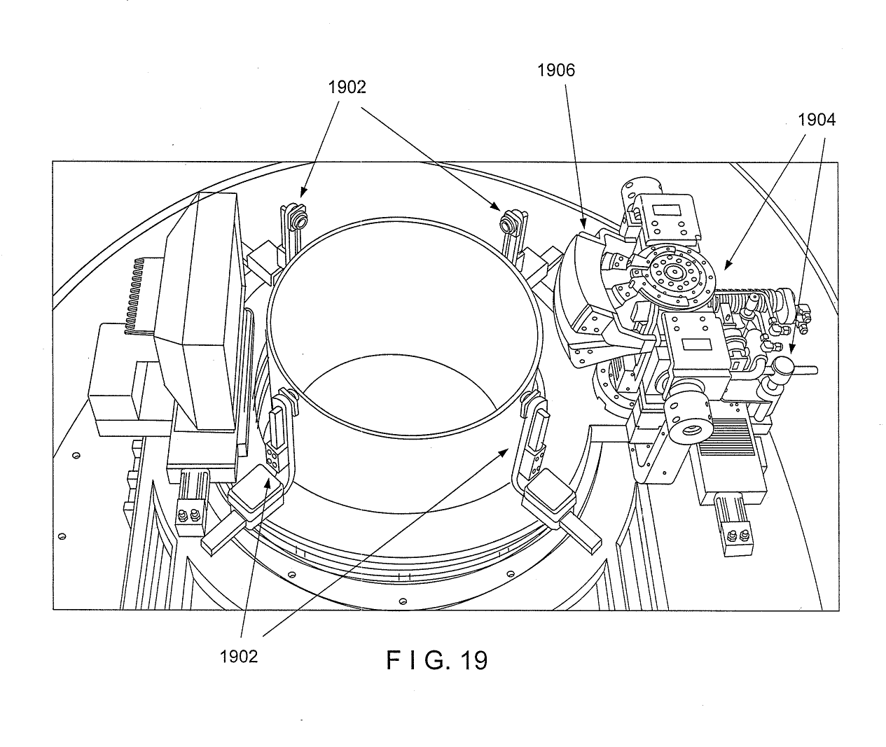

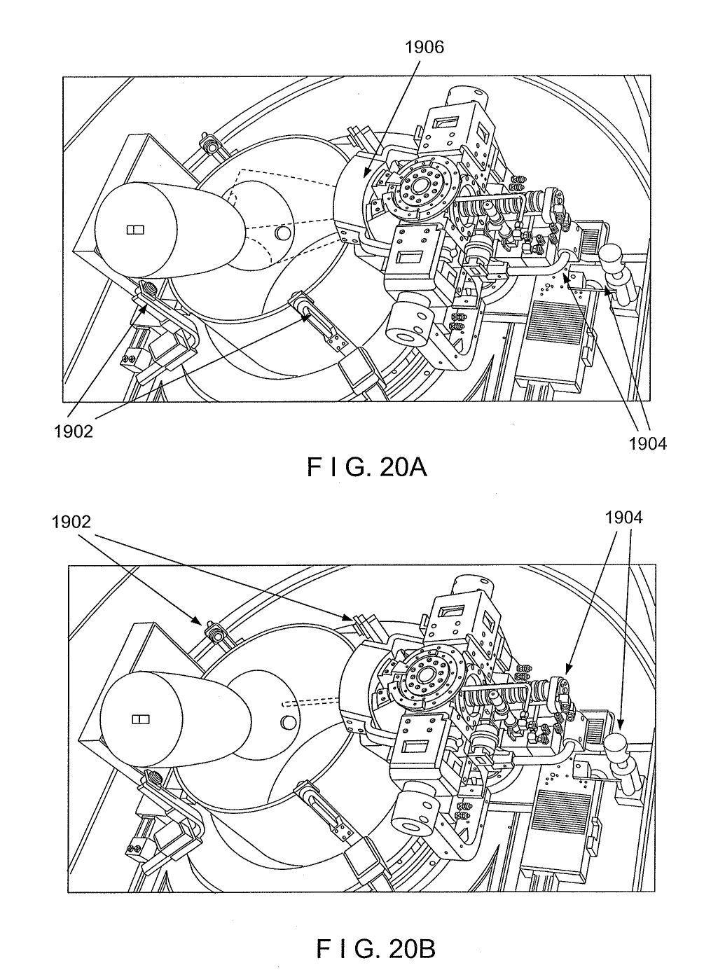

Additionally, the exemplary radiotherapy system 1802 can also include the exemplary tracking apparatus described above (and shown in, e.g., FIG. 7). As described above, the tracking apparatus can facilitate tracking movement of the organ being targeted during a radiotherapy treatment. Further, the radiotherapy system 1802 can include the exemplary collimator described above (and shown in FIGS. 8-17), which can facilitate shaping, aiming, and steering the radiotherapy beam or other radiation beams over the target area. Accordingly, the various exemplary embodiments of the present disclosure described above can be combined to provide a compact, robotic, beam-shaping radio therapy system that can be angled to accommodate various human anatomy. FIG. 19 shows an interior perspective view of the exemplary radiotherapy system 1802 of FIG. 18, including, for example, a collimator 1904 and an exemplary two-car configuration 1906. FIGS. 20A and 20B show an exemplary demonstration of the exemplary radiotherapy system 1802 in use. For example, FIG. 20A shows the exemplary radiotherapy system 1802 generating an imaging beam on a modeled patient, and FIG. 20B shows a treatment beam being generated onto a modeled patient.

Additionally, the exemplary radiotherapy system 1802 of FIG. 18 can include an optical surface tracking apparatus, as shown in FIG. 19. The exemplary optical surface tracking apparatus can include a plurality of optical surface tracking sensors 1902, e.g., three or four sensor radially disposed around an opening that can receive the human anatomy to be irradiated. The optical surface tracking apparatus can provide real-time optical surface tracking for patient set-up. Further, the exemplary radiotherapy system 1802 can include a shelf-shielding system. Specifically, the exemplary self-shielding system can contain the scattering and reflections of the radiation used during radiotherapy treatments. In one exemplary embodiment of the present disclosure, the shelf-shielding system can be implemented in both the construction of the exemplary collimated radiation source and the housing of the exemplary radiotherapy system 1802, which can thereby reduce and/or eliminate the need for costly and expensive vault construction.

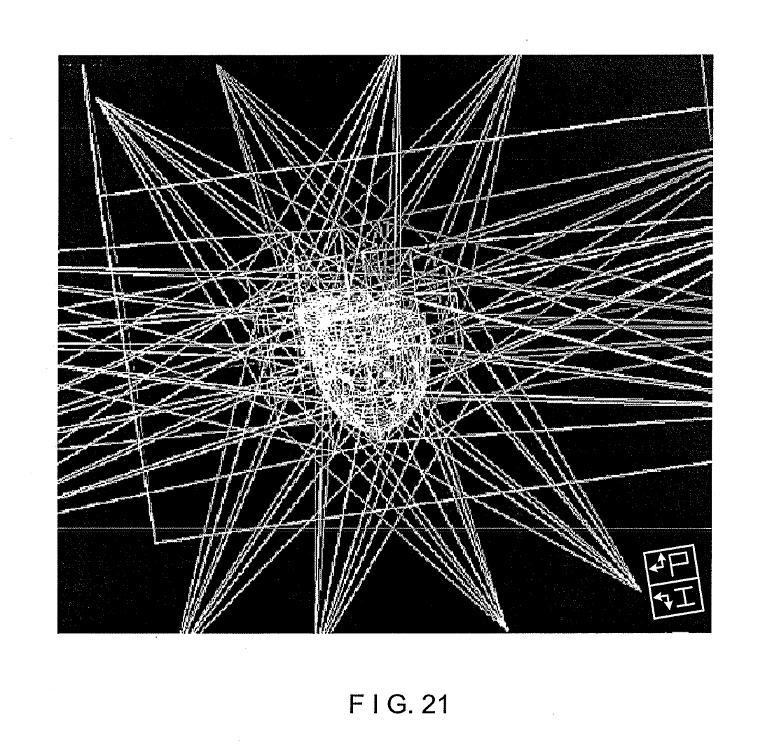

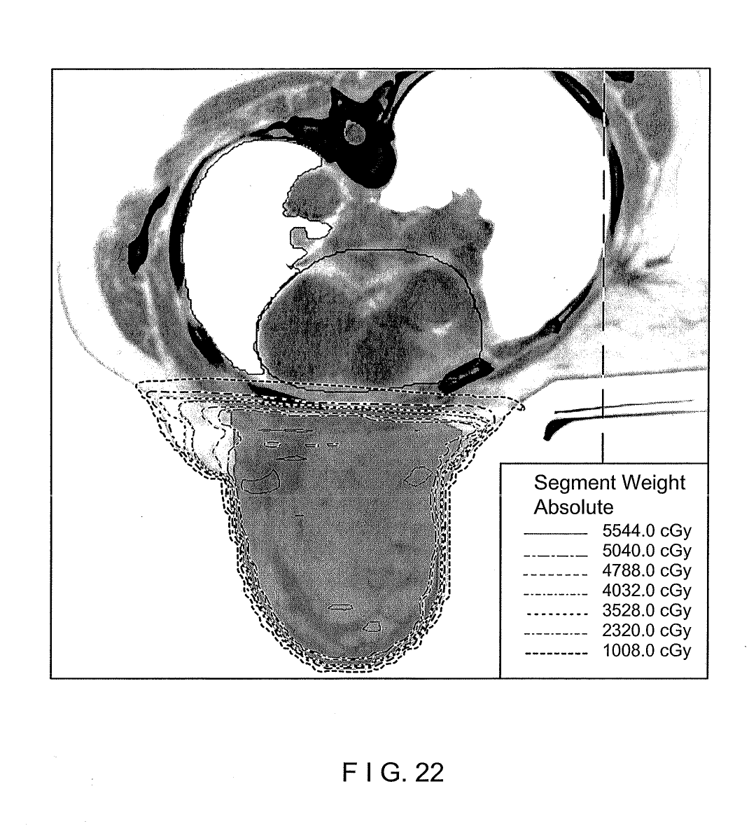

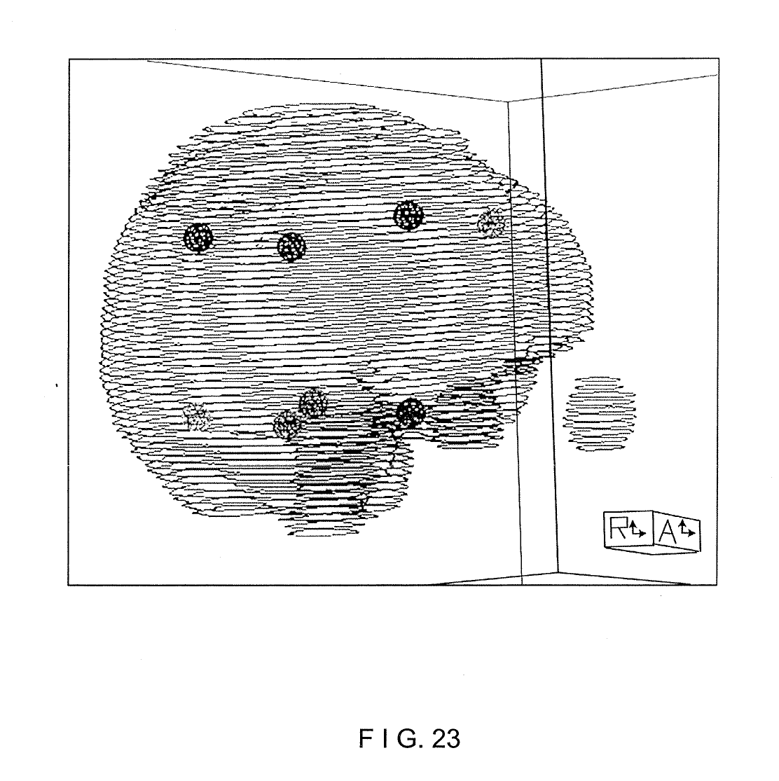

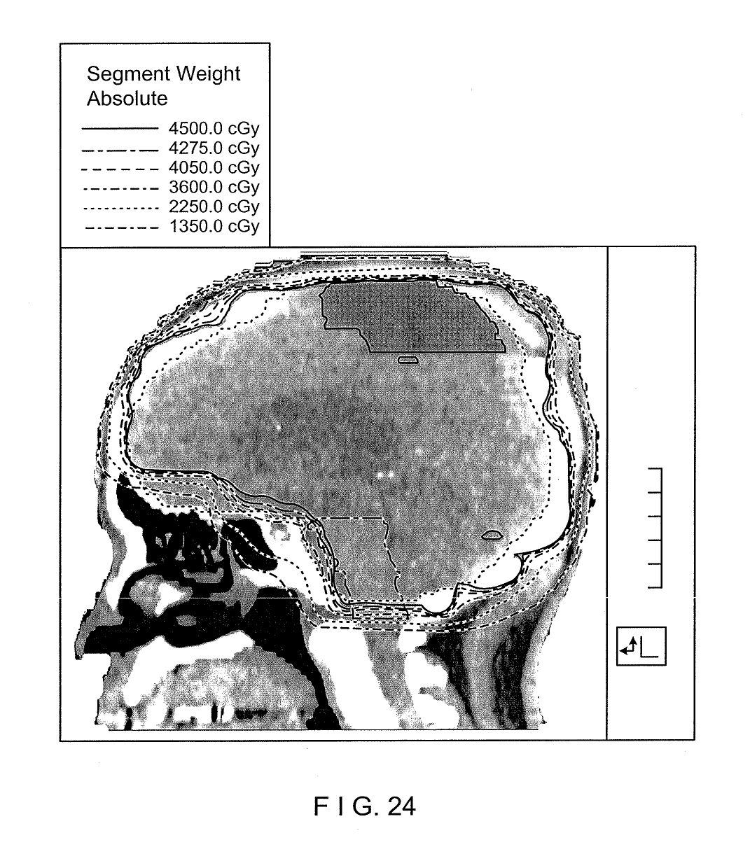

FIGS. 21-24 shows exemplary illustrations of a use and/or an implementation of the exemplary radiotherapy system 1802 of FIG. 18. For example, FIG. 21 shows an implementation of a non-coplanar breast treatment with a rendering of the breast and radiation generated by the exemplary radiotherapy system 1802 incident on the breast. FIG. 22 shows an exemplary rendering of the breast and a uniform dose distribution by unflattened beams performed by the exemplary radiotherapy system 1802 and with the radiotherapy system 1802 being oriented at about 0-degree angulation (e.g., parallel to the floor). FIG. 23 shows an illustration of a non-coplanar brain treatment in use, via an exemplary rendering of the brain and the targeting of the radiation to be generated by the exemplary radiotherapy system 1802 on the brain. FIG. 24 shows an exemplary rendering of the brain and a uniform dose distribution by unflattened beams performed by the exemplary radiotherapy system 1802 and with the radiotherapy system 1802 being oriented at about 60-degree angulation relative to the floor.

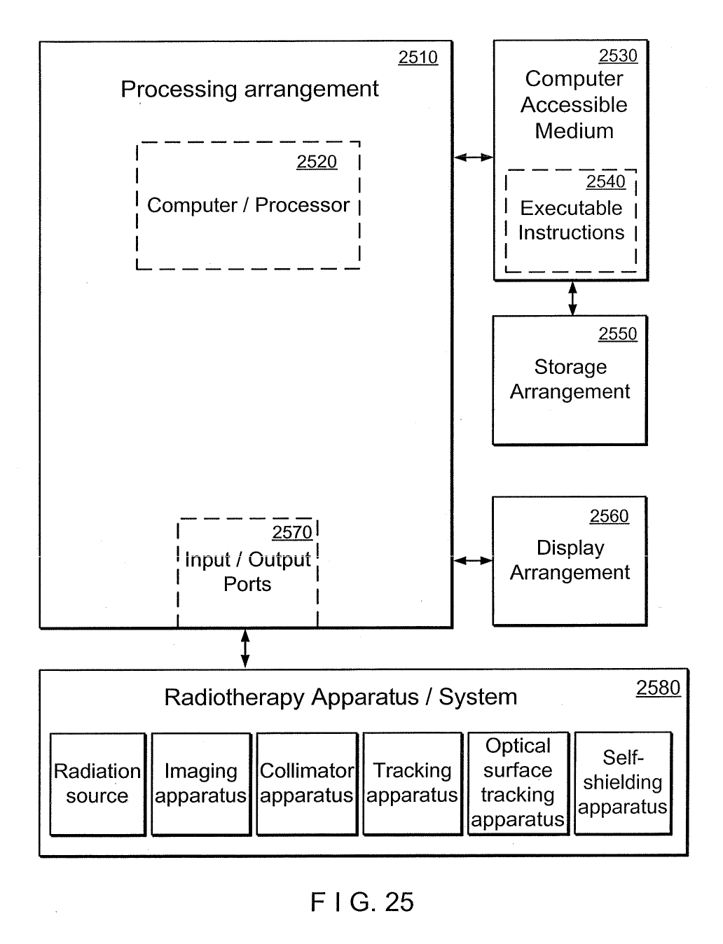

FIG. 25 shows an exemplary block diagram of an exemplary embodiment of a system according to the present disclosure. For example, exemplary procedures in accordance with the present disclosure described herein can be performed by or controlled using a radiotherapy apparatus/system 2580 and/or hardware processing arrangement and/or a computing arrangement 2510, separately and in conjunction with one another. Such exemplary processing/computing arrangement 2510 can be, e.g., entirely or a part of, or include, but not limited to, a computer/processor 2520 that can include, e.g., one or more microprocessors, and use instructions stored on a computer-accessible medium (e.g., RAM, ROM, hard drive, or other storage device).

As shown in FIG. 25, e.g., a computer-accessible medium 2530 (e.g., as described herein above, a storage device such as a hard disk, floppy disk, memory stick, CD-ROM, RAM, ROM, etc., or a collection thereof) can be provided (e.g., in communication with the processing arrangement 2510). The computer-accessible medium 2530 can contain executable instructions 2540 thereon. In addition or alternatively, a storage arrangement 2550 can be provided separately from the computer-accessible medium 2530, which can provide the instructions to the processing arrangement 2510 so as to configure the processing arrangement to execute certain exemplary procedures, processes and methods, as described herein above, for example.

Further, the exemplary processing arrangement 2510 can be provided with or include an input/output arrangement 2570, which can include, e.g., a wired network, a wireless network, the internet, an intranet, a data collection probe, a sensor, etc. As shown in FIG. 25, the exemplary processing arrangement 2510 can be in communication with an exemplary display arrangement 2560, which, according to certain exemplary embodiments of the present disclosure, can be a touch-screen configured for inputting information to the processing arrangement in addition to outputting information from the processing arrangement, for example. Further, the exemplary display 2560 and/or a storage arrangement 2550 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly being incorporated herein in its entirety. All publications referenced can be incorporated herein by reference in their entireties.

1-67. (canceled)

68. An apparatus for generating at least one radiation, comprising:
at least one radiation source arrangement that at least one of:
i. is pivotable about at least two axes and translatable on at least one track;
ii. disposed on the at least one track that is pivotable between a first position and a second position; or
iii. having a collimator assembly which is configured to move in a curved path so as to shape and direct the at least one radiation generated by the at least one radiation source arrangement.

69. The apparatus of claim 68, wherein the at least two axes are substantially orthogonal to one another.

70. The apparatus of claim 69, wherein one of the two axes is parallel to a tangent to a path of travel defined by the at least one track.

71. The apparatus of claim 70, wherein the at least one track includes a first rail and a second rail, and wherein the at least one radiation source arrangement includes a source arrangement disposed on the first rail and an aiming arrangement disposed on the second rail.

72. The apparatus of claim 71, wherein the aiming arrangement includes first and second motors configured to pivot the at least one radiation source arrangement about each of the at least two axes.

73. The apparatus of claim 68, wherein the at least one track at least one of (i) is pivotable between a first position and a second position, (ii) has a circular shape, or (iii) a non-circular shape.

74. The apparatus of claim 73, wherein an angle provided between a plane defined by the at least one track in the first position and a further plane defined by the at least one track in the second position is between about 0 degrees and about 90 degrees.

75. The apparatus of claim 68, wherein the at least one radiation includes at least one of (i) a radiotherapy radiation, or (ii) an associated energy of about 1 MV to about 6 MV.

76. The apparatus of claim 75, wherein at least two portions of the at least one track are substantially parallel to one another.

77. The apparatus of claim 68, wherein an angle provided between a plane defined by the at least one track in the first position and a further plane defined by the at least one track in the second position is between about 0 degrees and about 90 degrees.

78. The apparatus of claim 68, wherein the at least one radiation source arrangement is pivotable about at least two axes.

79. The apparatus of claim 68, wherein the collimator assembly includes a plurality of jaws movable about a first axis.

80. The apparatus of claim 79, wherein the collimator assembly includes a further plurality of jaws rotatable about a second axis.

81. The apparatus of claim 80, wherein each of the plurality of jaws and the further plurality of jaws is movable in a curved manner in opposite directions and simultaneously in a same direction.

82. The apparatus of claim 80, wherein the first and second axes are substantial orthogonal to one another.

83. The apparatus of claim 80, wherein the at least one radiation is provided through an opening created by the plurality of jaws and the further plurality of jaws.

84. An apparatus for tracking a target tissue, comprising:
a tracking assembly configured to track an internal movement of the target tissue within a living structure.

85. The apparatus of claim 84, wherein the tracking assembly at least one of (i) includes an ultrasonic imaging device configured to track the movement of the target tissue by continuously acquiring a position of a tissue associated with the target tissue, or (ii) provides the tracking information dynamically in real-time.

86. The apparatus of claim 85, wherein the tissue associated with the target tissue includes a diaphragm.

87. The apparatus of claim 84, further comprising at least one radiation source arrangement generating at least one radiation directed at the target tissue and directing the at least one radiation at the target tissue at least partially based on tracking information provided by the tracking assembly based on the movement.

88. The apparatus of claim 87, wherein the at least one radiation source arrangement is translatable on at least one track and is pivotable about at least two axes.

89. The apparatus of claim 88, wherein the at least one track at least one of (i) is pivotable between a first position and a second position, or (ii) includes a first rail and a second rail, and wherein the at least one radiation source arrangement includes a source arrangement disposed on the first rail and an aiming arrangement disposed on the second rail.

90. The apparatus of claim 88, wherein one of the two axes is parallel to a tangent to a path of travel defined by the at least one track.

91. The apparatus of claim 89, wherein the aiming arrangement includes first and second motors configured to pivot the at least one radiation source arrangement about each of the at least two axes.

92. The apparatus of claim 89, wherein an angle provided between a plane defined by the at least one track in the first position and a further plane defined by the at least one track in the second position is between about 0 degrees and about 90 degrees.

93. The apparatus of claim 84, further comprising a collimator assembly configured to move in a curved path so as to shape and direct the at least one radiation generated by the at least one radiation source arrangement.

94. The apparatus of claim 93, wherein the collimator assembly includes a plurality of jaws movable about a first axis.

95. The apparatus of claim 94, wherein the collimator assembly includes a further plurality of jaws rotatable about a second axis.

96. A system for generating at least one radiation, comprising:
at least one radiation source arrangement configured to generate the at least one radiation, is translatable on at least one track, and is pivotable about at least two axes;
a tracking assembly configured to track an internal movement of a target tissue within a living structure and provide tracking information to facilitate directing the at least one radiation generated by the at least one radiation source arrangement at the target tissue; and
a collimator assembly configured to move in a curved path so as to shape and direct the at least one radiation generated by the at least one radiation source arrangement.

97. The system of claim 96, wherein the at least one track at least one of:
(i) has a circular shape,
(ii) has a non-circular shape,
(iii) is pivotable between a first position and a second position, or
(iv) includes a first rail and a second rail, and wherein the at least one radiation source arrangement includes a source arrangement disposed on the first rail and an aiming arrangement disposed on the second rail.

98. The system of claim 97, wherein an angle provided between a plane defined by the at least one track in the first position and a further plane defined by the at least one track in the second position is between about 0 degrees and about 90 degrees.

99. The system of claim 98, wherein the at least two axes are substantially orthogonal to one another.

100. The system of claim 99, wherein one of the two axes is parallel to a tangent to a path of travel defined by the at least one track.

101. The system of claim 97, wherein the aiming arrangement includes first and second motors configured to pivot the at least one radiation source arrangement about each of the at least two axes.

102. The system of claim 97, wherein at least two portions of the at least one track are substantially parallel to one another.

103. The system of claim 96, wherein the collimator assembly includes a plurality of jaws rotatable about a first axis.

104. The apparatus of claim 103, wherein the collimator assembly includes a further plurality of jaws rotatable about a second axis.

105. The system of claim 104, wherein the first and second axes are substantial orthogonal to each other.

106. The system of claim 96, wherein the at least one radiation includes at least one of (i) a radiotherapy radiation, or (ii) an associated energy of about 1 MV to about 6 MV.

107. The system of claim 96, wherein the tracking assembly at least one of (i) provides tracking information dynamically in real-time, or (ii) includes an ultrasonic imaging device configured to track the movement of the target tissue by continuously acquiring a position of a tissue associated with the target tissue.

108. The system of claim 96, wherein the at least one radiation source arrangement generates the at least one radiation directed at the target tissue and directs the at least one radiation at the target tissue based on tracking information provided by the tracking assembly.

109. The system of claim 96, wherein the tissue associated with the target tissue includes a diaphragm.

110. The system of claim 96, further comprising an optical surface tracking arrangement.

111. The system of claim 110, wherein the optical surface tracking arrangement includes a plurality of optical sensors disposed radially around an opening configured to receive at least a portion of a patient.
